(19) United States
(12) Patent Application Publication
(10) Pub. No.: US 2013/0211137 A1
(43) Pub. Date: Aug. 15, 2013

(54) DIRECT ACTIVATION OF METHANE

(71) Applicant: KING ABDULLAH UNIVERSITY OF SCIENCE AND TECHNOLOGY, (US)

(72) Inventors: Jean-Marie BASSET, Thuwal (SA); Miao SUN, Thuwal (SA); Valerie CAPS, Thuwal (SA); Jeremie PELLETIER, Thuwal (SA); Edy ABOU-HAMAD, Thuwal (SA)

(73) Assignee: KING ABDULLAH UNIVERSITY OF SCIENCE AND TECHNOLOGY, Thuwal (SA)

(21) Appl. No.: 13/755,549

(22) Filed: Jan. 31, 2013

Related U.S. Application Data
(60) Provisional application No. 61/593,601, filed on Feb. 1, 2012.

(51) Int. Cl. C07C 51/16

(52) U.S. Cl. CPC C07C 51/16
USPC 562/607

(57) ABSTRACT

Heteropolyacids (HPAs) can activate methane at ambient temperature (e.g., 20° C.) and atmospheric pressure, and transform methane to acetic acid, in the absence of any noble metal such as Pd). The HPAs can be, for example, those with Keggin structure: H₄SiW₁₂O₄₀, H₃PW₁₂O₄₀, H₄SiMo₁₂O₄₀, or H₃PMo₁₂O₄₀, can be when supported on silica.

This application claims priority to U.S. Patent Application No. 61/593,601, filed Feb. 1, 2012, which is hereby incorporated by reference in its entirety.

### TECHNICAL FIELD

This invention relates to direct activation of methane to products, such as, acetic acid.

### BACKGROUND

Many industrial uses of gaseous hydrocarbons such as methane, ethane, and ethene, require first converting the gas to a second compound. The second compound can desirably be a liquid, and sometimes has more carbon atoms than the precursor gas. Such conversions require the use of a catalyst. Current catalysts suffer from disadvantages such as requiring elevated temperatures (e.g., above 100° C., 200° C., 300° C., 400° C., 500° C., or even higher); requiring elevated pressure (e.g., well above ambient pressure, such as above 10 atm, 20 atm, or 30 atm, or even higher); and producing large amounts of undesired products such as CO₂. As the amount of available natural gas reserves surpasses the amount of available petroleum, use of methane is growing in economic importance, particularly for uses other than burning methane as fuel.

### SUMMARY

Methane can be directly converted to acetic acid at ambient temperature and atmospheric pressure. This method allows production of acetic acid from a widely accessible resource (methane, instead of methanol, ethene or ethane) in an energy-efficient process. Furthermore, this direct, solid/gas process, from methane to acetic acid is green (minimum number of steps, minimum energy input required, no need to use expensive noble metals).

In one aspect, a method for converting methane to acetic acid includes contacting methane with a heteropolyacid. The conversion can take place at a temperature no greater than 500° C., no greater than 400° C., no greater than 300° C., no greater than 200° C., no greater than 100° C., or lower. Conversion can take place at ambient temperature. Conversion can take place at a pressure no greater than 100 bar, no greater than 50 bar, no greater than 30 bar, no greater than 20 bar, no greater than 10 bar, or lower. Conversion can take place at atmospheric pressure.

The heteropolyacid can be substantially free of noble metals. The method can include heating the heteropolyacid after contacting, thereby releasing acetic acid. The heteropolyacid can include H₄SiW₁₂O₄₀, H₃PW₁₂O₄₀, H₄SiMo₁₂O₄₀, or H₃PMo₁₂O₄₀. The heteropolyacid can be associated with a solid support. The solid support can include a silicon oxide. The method can include separating the acetic acid from the heteropolyacid. The method can include contacting the heteropolyacid with an oxidant.

Other aspects, embodiments, and features will be apparent from the following description, the drawings, and the claims.

### BRIEF DESCRIPTION OF THE DRAWINGS

FIG. 1 schematically illustrates conversion of methane to acetic acid by HPAs.

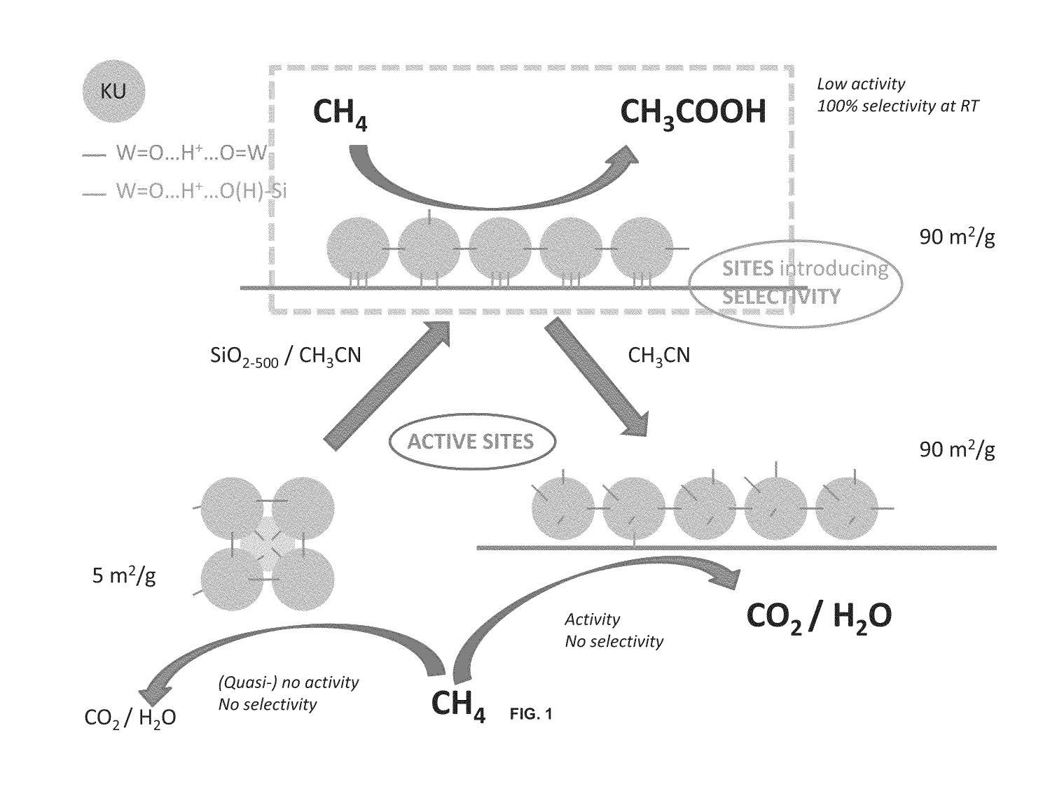

FIG. 2 illustrates a proposed mechanism for conversion of methane to acetic acid involving superacidic H.

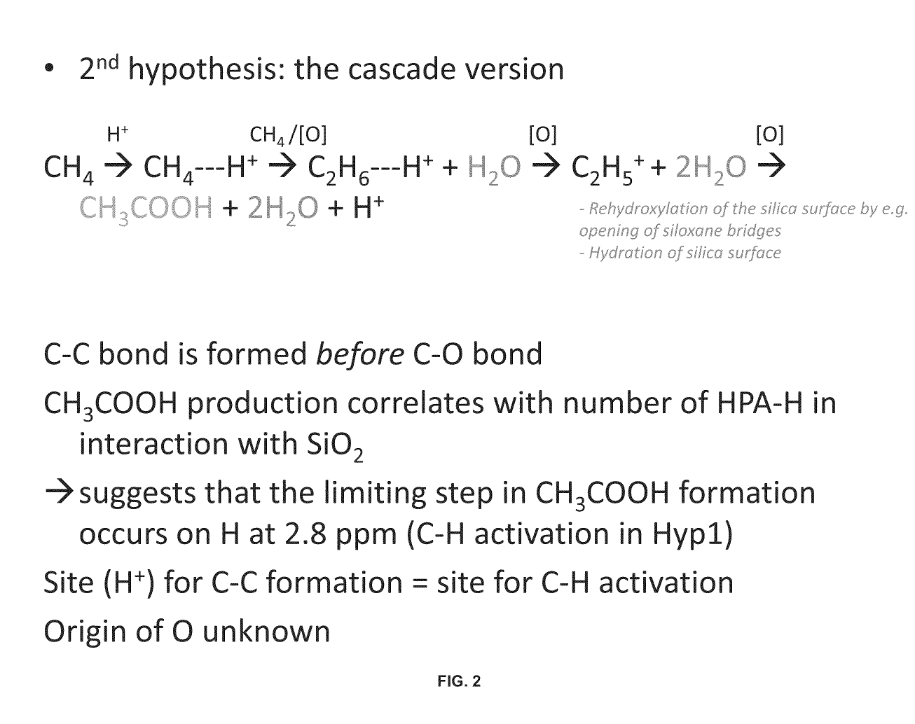

FIG. 3 illustrates a proposed mechanism involving superacidic H⁺ for conversion of methane to acetic acid.

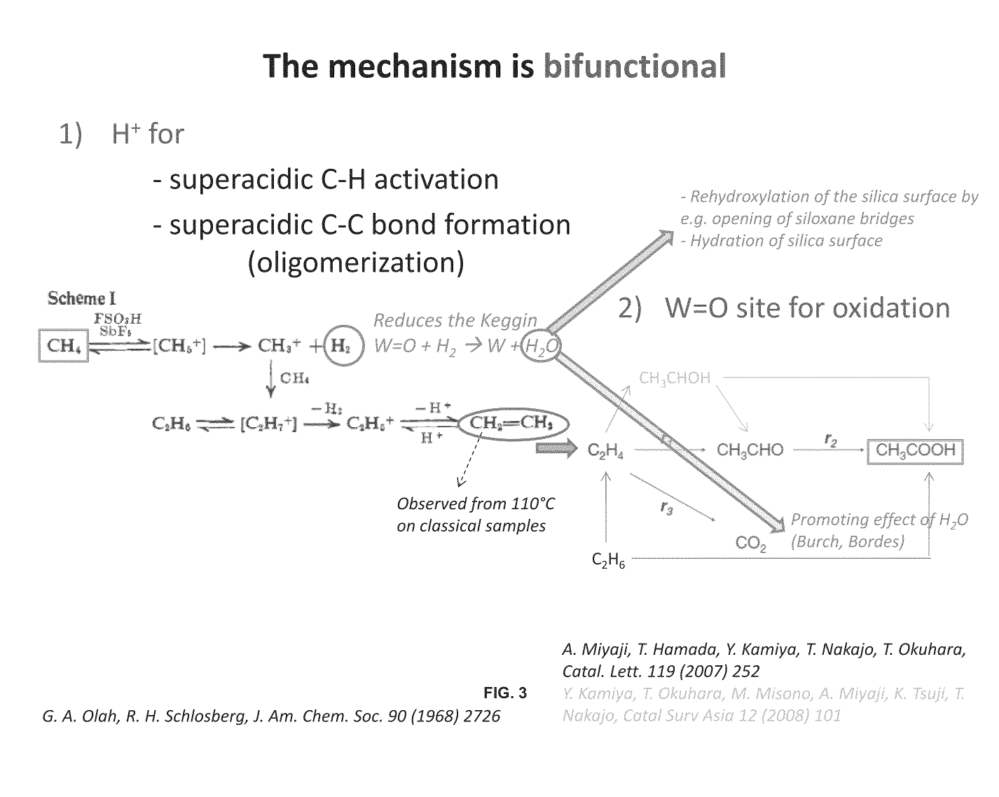

FIG. 4 is a graph showing normalized activity (total number of molecules produced in the gas phase divided by the total number of HPA protons present).

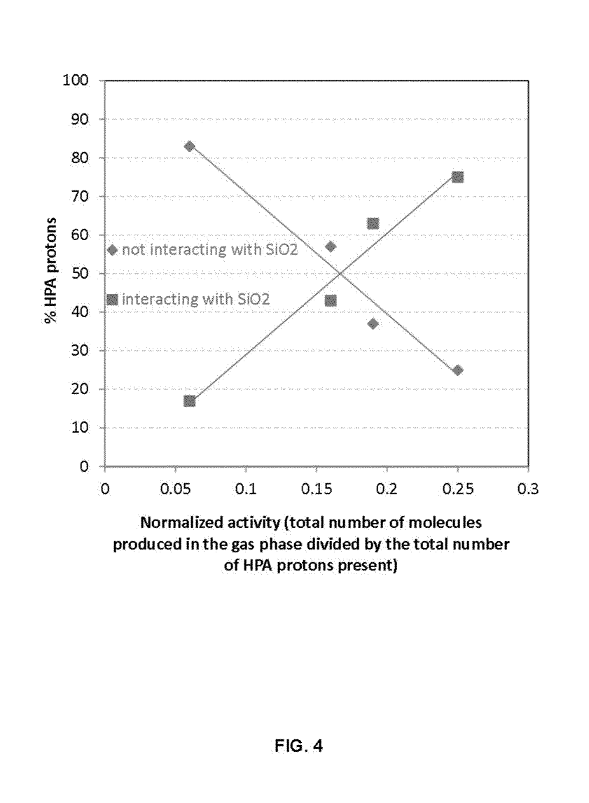

### DETAILED DESCRIPTION

The tungsto-silicic heteropolyacid (HPA) with Keggin structure H₄SiW₁₂O₄₀ is currently being used in the Showa-Denko process as a solid catalyst for direct acetic acid production from ethene (I. D. Dobson, Green Chem. 5 (2003) G78, which is incorporated by reference in its entirety). At the molecular level, this transformation is thought to involve a Wacker-type mechanism, in which activation of the ethylenic carbon-hydrogen bond occurs on the Pd²⁺ component of the silica-supported catalyst. In this model, the HPA is proposed to act essentially as a co-catalyst by regenerating the active Pd site. In the absence of Pd, those supported HPAs are reported to be inactive for the formation of acetic acid (K. G. Fang, X. P. Wang, J. L. Zhang, T. X. Cai, Chinese Chem. Lett. 12 (2001) 125-126, which is incorporated by reference in its entirety). However, low yields of acetic acid can be obtained directly from ethane on Pd-free supported molybdo(vanado)phosphoric heteropolyacids (M. Sopa, A. W [symbol] c/law-Held, M. Grossy, J. Pijanka, K. Nowińska, Applied Catalysis A: General 285 (2005) 119-125, which is incorporated by reference in its entirety), suggesting that supported HPAs can activate secondary aliphatic C—H bonds (and the formation of secC—O bond), even in the absence of Pd or other noble metals.

On the other hand, the activation of methane by HPAs and supported HPAs is generally performed at the higher temperatures required to activate the more stable C—H bond (439 kJ/mol). Temperatures higher than 500° C. are typically required. In the presence of oxidizing agents stronger than molecular oxygen (e.g., N₂O or H₂O₂), low yields of formaldehyde, formic acid, methanol products can be readily produced in the gas phase from 300° C., together with large amounts of CO and CO₂. Activating the high energy primary C—H bond of methane under milder conditions (ambient temperature and pressure) is scientifically challenging.

Biological enzymes of the methane monooxygenase type need oxygen, water and a co-factor to activate methane under these conditions of temperature and pressure. See, e.g., Merkx, M., et al., Dioxygen Activation and Methane Hydroxylation by Soluble Methane Monooxygenase: A Tale of Two Irons and Three Proteins, Angew. Chemie 40, 15 (2001), which is incorporated by reference in its entirety. Liquid superacids can achieve it at atmospheric pressure but above 100° C. only, while the Shilov systems (soluble Pt(II) salts) operating at about 100° C. require elevated pressure (30 atm). See, for example, G. A. Olah, R. H. Schlosberg, J. Am. Chem. Soc. 90, 2726 (1968); G. A. Olah, G. Klopman, R. H. Schlosberg, J. Am. Chem. Soc. 91, 3261 (1969); A. E. Shilov, Activation of Saturated Hydrocarbons by Transition Metal Complexes (Reidel, Dordrecht, 1984); M. Lersch, M. Tilset, Chem. Rev. 105, 2471 (2005); and J. L. Look, U. Fekl, K. I. Goldberg, Activation and Functionalization of C—H Bonds, in: ACS Symposium Series 885 (Eds.: Karen I. Goldberg and Alan S. Goldman), 283 (2004); each of which is incorporated by reference in its entirety.

Previously, the only system for direct methane-to-acetic acid was the so-called Catalytica-Periana Pd (II) complex operating at 30 atm/180° C. in concentrated sulfuric acid. This system leads to CH₃COOH, in addition to CH₃COOSO₃H and CH₃OH. See R. A. Periana, O. Mironov, D. Taube, G. Bhalla, C. J. Jones, Science 301, 814 (2003), which is incorporated by reference in its entirety. Methanol is identified as a reactive intermediate which triggers carbonylation of a Pd—CH₃ species via CO species generated in-situ from its slow over-oxidation. The reaction does not occur in the absence of Pd.

It has now been discovered that HPAs can activate methane at ambient temperature (20° C.) and atmospheric pressure (1 atm) in a clean solid-gas reaction. The HPA can have a Keggin structure. The reaction can be carried out under ambient conditions of temperature and pressure, or can be carried out with a temperature and/or pressure above or below ambient. The HPA can be located on a support, such as a silicon oxide like silica. Exemplary HPAs useful for direct conversion of methane to acetic acid include H₄SiW₁₂O₄₀, H₃PW₁₂O₄₀, H₄SiMo₁₂O₄₀, and H₃PMo₁₂O₄₀. In some cases, the interaction between the HPA and a silica support can be important for methane-to-acetic acid conversion, e.g., the interaction of protons with silica oxygen atoms and HPA oxygen atoms. Such an interaction can facilitate, promote, or enhance the superacidic nature of the HPA proton. The HPA can be substantially free of Pd or other noble metals. “Substantially free” can mean that no noble metal has been added to the HPA.

The catalyst can be on a solid support. The catalyst can be deposited on a surface of the solid support, covalently bonded to the surface of the solid support, or entrapped within the solid support. The solid support can, for example, be chosen from metal oxides, refractory oxides and molecular sieves, in particular from silicon oxides (e.g., silica), aluminum oxides, zeolites, clays, titanium oxide, cerium oxide, magnesium oxide, niobium oxide, zinc oxide, molybdenum oxide, iron oxide, cobalt oxide, tantalum oxide or zirconium oxide.

### Examples

It has been shown that when supported on silica, HPAs with Keggin structure (such as H₄SiW₁₂O₄₀, H₃PW₁₂O₄₀, H₄SiMo₁₂O₄₀, H₃PMo₁₂O₄₀) activated methane at ambient temperature (20° C.) and atmospheric pressure (1 atm) in a clean solid-gas reaction. Furthermore, the low temperature reaction between methane and silica-supported HPA led, in a single step, to the formation of acetic acid.

In this Pd-free system, acetic acid was the only observed carbonaceous product formed by the low temperature reaction of methane with the solid surface, as evidenced by ¹³C CP/MAS solid-state NMR. The ¹³C NMR spectrum of H₄SiW₁₂O₄₀/SiO₂₋₅₀₀ contacted with flowing ¹³CH₄ (100%, 5 mL/min, fixed-bed reactor configuration) for 30 min at 25° C. exhibited chemical shifts at 20 ppm (—CH₃) and 182 ppm (—COO), which are characteristic of methyl acetate/acetic acid As the temperature of reaction was increased to 80° C. and then 120° C., these signals grew, allowing 2D HETCOR signals to be acquired, confirming the peak attributions. The 2D HETCOR spectrum obtained after reaction at 120° C. displayed the correlation between the carbon signal at 20 ppm (—CH₃) and the proton signal at 2.0±0.1 ppm (—CH₃), which was expected from the methyl group (FIG. 6A). The ¹H signal at 2 ppm (—CH₃) also correlated with the ¹³C signal at 182 ppm (—COO), as observed by 2D long distance HETCOR, confirming that the —CH₃ and —COO functionalities belonged to the same molecule.

As methyl acetate/acetic acid was produced on the material surface, no gaseous products were observed. Chromatograms obtained on all 4 channels of the micro-GC were indeed free of peaks, except for that corresponding to the CH₄ reactant. Hence, no CO, CO₂, alkane, alkene, or other oxygenated products were formed, whether on the solid or in the gas phase, when CH₄ reacted with silica-supported HPA to give acetic acid below 100° C.

The HPAs/silica interface underwent significant changes in the course of this reaction, as evidenced by Magic Angle Spinning ¹H solid-state NMR. This study was performed on the H₄SiW₁₂O₄₀/SiO₂₋₅₀₀ sample. Before reaction with methane, the ¹H NMR spectrum of H₄SiW₁₂O₄₀/SiO₂₋₅₀₀ exhibited 3 characteristic features, at 1.8, 2.8 and 9.9 ppm, which corresponded to unreacted silanols, protonated silanols, and HPA protons, respectively. The peak at 1.8 ppm was indeed the main feature of the ¹H NMR spectrum of SiO₂₋₅₀₀ before HPA deposition; it is characteristic of isolated silanols. This peak shows that, in H₄SiW₁₂O₄₀/SiO₂₋₅₀₀, some silanols of the silica surface were not perturbed by the presence of the HPA (have not reacted upon HPA grafting). The broad peak at 9.9 ppm was very similar to the single signal observed in the ¹H NMR spectrum of unsupported, dehydrated HPA (9.5 ppm); it was consistent with protons of the HPA which are free from interaction with the silica surface. Finally, a new peak at 2.8 ppm, which was absent from the ¹H NMR spectra of either SiO₂₋₅₀₀ or HPA alone, was consistent with HPA protons in close interaction with the silica surface and in particular with silanol groups. This was evidenced by an auto-correlation in the double quantum (DQ) ¹H-¹H NMR spectrum. An auto-correlation in the triple quantum (TQ) spectrum indicated that at least three equivalent protons were involved in the HPA/silica interface. On the other hand, the self-correlation of the signal at 9.9 ppm was only observed in the DQ spectrum and is absent from the TQ spectrum. This suggested that two protons of each H₄SiW₁₂O₄₀ unit were free from interaction with the silica surface and that the other two were involved in interaction with surface silanols.

Acetic acid desorbed from the surface at about 200° C. or above.

TABLE 1
Data from gas-phase analysis (micro-GC)

| | | | Selectivity | | |
|---|---|---|---|---|---|
| material | Ratio¹ | Activity² | CH₃COOH | CO₂ | H₂ |
| H₄SiW₁₂O₄₀/ | 0.64 | 0.46 | 24 | 63 | 13 |
| SiO₂₋₅₀₀ | | | | | |
| H₄SiW₁₂O₄₀/ | 0.92 | 0.23 | 33 | 55 | 12 |
| SiO₂₋₂₅ | | | | | |
| H₄SiMo₁₂O₄₀/ | 0.24 | 0.06 | 42 | 29 | 29 |
| SiO₂₋₅₀₀ | | | | | |
| H₄SiMo₁₂O₄₀/ | 0.64 | 0.16 | 48 | 40 | 11 |
| SiO₂₋₂₅ | | | | | |
| H₃PMo₁₂O₄₀/ | 0.75 | 0.25 | 35 | 65 | 0 |
| SiO₂₋₅₀₀ | | | | | |
| H₃PMo₁₂O₄₀/ | 0.69 | 0.23 | 43 | 51 | 7 |
| SiO₂₋₂₅ | | | | | |
| H₃PMo₁₂O₄₀/ | 0.57 | 0.19 | 4 | 79 (96) | 18 (0) |
| SiO₂₋₅₀₀ | | | | | |
| (H₂ release > | | | | | |
| 340° C.) | | | | | |
| H₃PMo₁₂O₄₀/ | 0.42 | 0.14 | 59 | 16 | 24 |
| SiO₂₋₂₅ | | | | | |
| (H₂ release > | | | | | |
| 300° C.) | | | | | |

¹Total nb of molecules produced at T < 350° C./nb of HPA present in the system
²Total nb of molecules produced at T < 350° C. per proton (normalized activity)

TABLE 2
Data from analysis of the material (solid-state ¹H NMR)

| | | % HPA protons |
|---|---|---|
| | % H of the system involved | interacting |
| material | in the HPA/SiO₂ interface | with SiO₂ |
| H₄SiW₁₂O₄₀/SiO₂₋₅₀₀ | 51 | 43 |
| H₄SiMo₁₂O₄₀/SiO₂₋₅₀₀ | 24 | 17 |
| H₃PMo₁₂O₄₀/SiO₂₋₅₀₀ | 76 | 75 |
| H₃PMo₁₂O₄₀/SiO₂₋₅₀₀ | 60 | 63 |
| (H₂ release > 340° C.) | | |

TABLE 3
Data from gas phase analysis - temperature at which release occurs from
the material surface to the gas phase (° C.)

| material | CH₃COOH | CO₂ | H₂ |
|---|---|---|---|
| H₄SiW₁₂O₄₀/SiO₂₋₅₀₀ | 200 | 200 | 120 |
| H₄SiW₁₂O₄₀/SiO₂₋₂₅ | 200 | 200 | 150 |
| H₄SiMo₁₂O₄₀/SiO₂₋₅₀₀ | 200 | 200 | 200 |
| H₄SiMo₁₂O₄₀/SiO₂₋₂₅ | 200 | ca. 200 | 100 |
| H₃PMo₁₂O₄₀/SiO₂₋₅₀₀ | 200 | 200 | — |
| H₃PMo₁₂O₄₀/SiO₂₋₂₅ | 200 | ca. 200 | 120 |
| H₃PMo₁₂O₄₀/SiO₂₋₅₀₀ | 200 | 200 | 340 |
| (H₂ release > 340° C.) | | | |
| H₃PMo₁₂O₄₀/SiO₂₋₂₅ | 200 | ca. 200 | 300 |
| (H₂ release > 300° C.) | | | |

### EXPERIMENTAL

Materials: Preparation of silica-supported H₄SiW₁₂O₄₀ began with partial dehydroxylation of the silica support (99.8%, Sigma-Aldrich) under vacuum (<3×10⁻⁵ bar) at 500° C. for 12 h to yield SiO₂₋₅₀₀. Separately the silico-tungstic Keggin HPA was dehydrated under high vacuum at 100° C. for 1 h then at 200° C. for 1 h. Most of the crystallization water (6-7%, as measured by TGA) was thus removed before solubilization of the HPA in dried acetonitrile and contact with SiO₂₋₅₀₀. All manipulations were carried out under Ar. The resulting H₄SiW₁₂O₄₀/SiO₂₋₅₀₀ powder was isolated after solvent removal (under vacuum) and dried at room temperature under vacuum (<3×10⁻⁵ bar), and then stored under argon. The surface area of H₄SiW₁₂O₄₀/SiO₂₋₅₀₀ was 93 m²/g.

Methane Activation: H₄SiW₁₂O₄₀/SiO₂₋₅₀₀ was contacted with a mixture of 10% ¹³CH₄/90% ¹²CH₄ (total flow rate of 5 mL/min), and in some cases heated, and the reaction was quenched at a given temperature below 195° C. (temperature at which acetic acid is evolved), except for the experiment at 25° C.

CP/MAS¹³C NMR: The following sequence was used: 90° pulse on the proton (pulse length 2.4 μs), then a cross-polarization step with a contact time typically set to 5 ms, and finally acquisition of the ¹³C signal under high power proton decoupling. The delay between the scan was set to 5 s, to allow the complete relaxation of the ¹H nuclei. An apodization function (exponential) corresponding to a line broadening of 80 Hz was applied.

2D HETCOR: The 2D ¹H-¹³C chemical shift correlation solid-state NMR spectroscopy experiments were conducted on a Bruker DSX-400 spectrometer using a 3.2 mm MAS probe. For the cross-polarization step, a ramped radio frequency (RF) field centered at 75 KHz was applied to protons, while the carbon RF field was matched to obtain optimal signal. A total of 64 t₁ increments with 10 000 scans each were collected. The sample spinning frequency was 8.5 kHz, and the contact time for the cross-polarization step was set to 0.4 ms allows the selective observation of C—H nuclei which are spatially very close. For long distance 2D ¹H-¹³C chemical shift correlation solid-state NMR spectroscopy experiment, longer contact times (5 ms) were used. Extra correlation peaks were observed which arose from longer range dipolar through-space interactions.

Other embodiments are within the scope of the following claims.

What is claimed is:

1. A method for converting methane to acetic acid comprising contacting methane with a heteropolyacid.

2. The method of claim 1, wherein conversion takes place at a temperature no greater than 100° C.

3. The method of any of the preceding claims, wherein conversion takes place at ambient temperature.

4. The method of any of the preceding claims, wherein conversion takes place at a pressure no greater than 30 bar.

5. The method of any of the preceding claims, wherein conversion takes place at atmospheric pressure.

6. The method of any of the preceding claims, wherein the heteropolyacid is substantially free of noble metals.

7. The method of any of the preceding claims, further comprising heating the heteropolyacid after contacting, thereby releasing acetic acid.

8. The method of any of the preceding claims, wherein the heteropolyacid includes H₄SiW₁₂O₄₀, H₃PW₁₂O₄₀, H₄SiMo₁₂O₄₀, or H₃PMo₁₂O₄₀.

9. The method of any of the preceding claims, wherein the heteropolyacid is associated with a solid support.

10. The method of any of the preceding claims, wherein the solid support includes a silicon oxide.

11. The method of any of the preceding claims, further comprising separating the acetic acid from the heteropolyacid.

12. The method of any of the preceding claims, further comprising contacting the heteropolyacid with an oxidant.
